(19) United States
(12) Patent Application Publication
(10) Pub. No.: US 2018/0055412 A1
(43) Pub. Date: Mar. 1, 2018

(54) NEONATE FLOW SENSOR

(71) Applicant: OrangeMed, Inc., Irvine, CA (US)

(72) Inventors: Clayton R. Platt, Lake Forest, CA (US); Germano Forti, Irvine, CA (US); Richard William Crawford, JR., Yucaipa, CA (US)

(73) Assignee: OrangeMed, Inc., Irvine, CA (US)

(21) Appl. No.: 15/675,546

(22) Filed: Aug. 11, 2017

Related U.S. Application Data
(60) Provisional application No. 62/379,653, filed on Aug. 25, 2016.

(51) Int. Cl. A61B 5/087; A61B 5/097

(52) U.S. Cl. CPC A61B 5/087; A61B 2503/045; A61B 5/097

(57) ABSTRACT

A flow sensor comprising a tubular body having a bore, a ventilator end, a patient end, and a central region; a restrictor assembly located adjacent the central region, and fluidly connected to the bore, the restrictor assembly having a first pressure port having a first diameter, proximate the patient end, and fluidly connected to a second portion of a first lumen; a second pressure port having a second diameter, proximate the ventilator end, and fluidly connected to a second portion of a second lumen; and a restrictor post having a length connected to a shaft having a width; wherein a first portion of the first lumen is perpendicular to a second portion of the first lumen, and a first portion of the second lumen is perpendicular to a second portion of the second lumen.

This application claims priority to U.S. Provisional Application No. 62/379,653 filed Aug. 25, 2016, which application is incorporated herein by reference, in its entirety, for any purpose.

### TECHNICAL FIELD

This disclosure relates generally to flow sensors, and examples of methods and apparatus for neonatal flow sensors are described. Example sensors may find use with mechanical ventilation systems.

Neonatal flow sensors may use a restrictor post as a flow restriction to form a differential pressure which changes proportionally to the flow rate. By measuring the differential pressure, the flow rate of the airflow into and out of a patient can be determined. A fixed orifice differential pressure sensor may be used for measuring bi-directional flow in neonate patient airways. A typical sensor may be bulky, heavy, and increase dead space during patient use due to the spacing of standard lumens surrounding the restrictor post, resulting in discomfort to the patient and inaccurate measurements in low flow applications. The sensors may also provide for inaccurate measurements as mucus or condensation may cling to the restrictor post or internal tubing transmitting the pressure signals and influence the overall measurement.

The information included in this Background section of the specification, including any references cited herein and any description or discussion thereof, is included for technical reference purposes only and is not to be regarded subject matter by which the scope of the invention as defined in the claims is to be bound.

### SUMMARY

In an example, a flow sensor may have a first tubular body having a first bore, a first central axis, and a patient end configured to be in fluid communication with a ventilator; a second tubular body having a second bore, a second central axis, and a ventilator end confirmed to be in fluid communication with a ventilator; a third tubular body having a third bore and a third central axis, wherein the third bore is adjacent both the first bore and the second bore to form a continuous central bore extending from the patient end to the ventilator end; a connector comprising: a first pressure port adjacent the first tubular body and in fluid communication with a first end of a first lumen; a second pressure port adjacent the second tubular body and in fluid communication with a first end of a second lumen; and a restrictor having a post and a shaft, the shaft having a width equal to a spacing between the first and the second pressure ports and the post having a length that is aligned with the continuous central bore; wherein the first end of the first lumen is perpendicular to a second end of the first lumen, and the first end of the second lumen is perpendicular to a second end of the second lumen.

In an example, a flow sensor may comprise a tubular body having a bore, a ventilator end, a patient end, and a central region; a restrictor assembly located adjacent the central region and fluidly connected to the bore, the restrictor assembly having a first pressure port having a first diameter, proximate the patient end, and fluidly connected to a second portion of a first lumen; a second pressure port having a second diameter, proximate the ventilator end, and fluidly connected to a second portion of a second lumen; and a restrictor post having a length connected to a shaft having a width; wherein a first portion of the first lumen is perpendicular to a second portion of the first lumen, and a first portion of the second lumen is perpendicular to a second portion of the second lumen.

In an example, a neonate flow sensor may comprise: a connector having a first lumen with a first end fluidly connected to a first pressure port and a second end perpendicular to the first lumen's first end; a second lumen with a first end fluidly connected to a second pressure port and a second end perpendicular to the second lumen's first end; and a restrictor having a shaft connected to a post, the shaft located between the first lumen's first end and the second lumen's first end; and a tubular body with a patient end, a ventilator end, and a bore, all fluidly connected to the first and second lumens, and the second end of the first lumen and the second end of the second lumen are proximate the ventilator end.

### BRIEF DESCRIPTION OF THE DRAWINGS

FIG. 1 is a schematic view of a respiratory system using a neonatal flow sensor,

FIG. 2 is a perspective view of an example neonatal flow sensor.

FIG. 3 is a front elevation view of the flow sensor of FIG. 2.

FIG. 4 is a rear elevation view of the flow sensor of FIG. 2.

FIG. 5 is a cross-sectional isometric view of the neonatal flow sensor of FIG. 2 along line 4-4.

FIG. 6 is an enlarged cross-sectional right side elevation view of the flow sensor of FIG. 5.

### DETAILED DESCRIPTION

A neonatal patient may have shallow, low flow respirations that are difficult to detect and measure. Neonatal flow sensors may use a restrictor post as a flow restriction to form a differential pressure which changes proportionally to the flow rate. By measuring the differential pressure, the flow rate of the airflow into and out of a patient can be determined. Existing flow sensors using a restrictor post may be large, bulky, and create additional air dead space during use, therefor decreasing patient comfort and flow measurement efficiency.

Example flow sensors described herein may include a shape and position of lumens within the connector which may overcome and/or help address these or other issues, which may enhance patient comfort and measurement accuracy in some examples. Example flow sensors may have a restrictor with a pair of lumens with first ends that are generally perpendicular to second ends that allow the center of the mass of the restrictor, and therefore the flow sensor, to be biased away from the patient. The shift of the center of mass may increase patient comfort, help with alignment of the flow sensor with reference to the ventilator and patient respiration system, and allow the accuracy of the flow sensor to increase when measuring gaseous flow through the flow sensor. In addition, the compact design may also allow the flow sensor to be placed in a closer proximity to the patient, and reduce the additional dead space which may increase the accuracy of the sensor since it may better detect measure the inhalations and exhalations of the patient.

Example advantages described herein are provided to facilitate understanding. It is to be understood that not all examples may address all, or even any, of the shortcomings of previous systems, and that not all examples may have all, or even any, of the described advantages.

Example flow sensors may increase the accuracy of flow measurement because of the layout of the pressure ports that will more accurately determine the disruption of the flow caused by the flow being forced around the restrictor post. The example flow sensors may also increase the accuracy of flow measurement as moist excretions that may coat the inside of the valve and undesirably disrupt the flow measurement may be drained through a sloped continuous central bore.

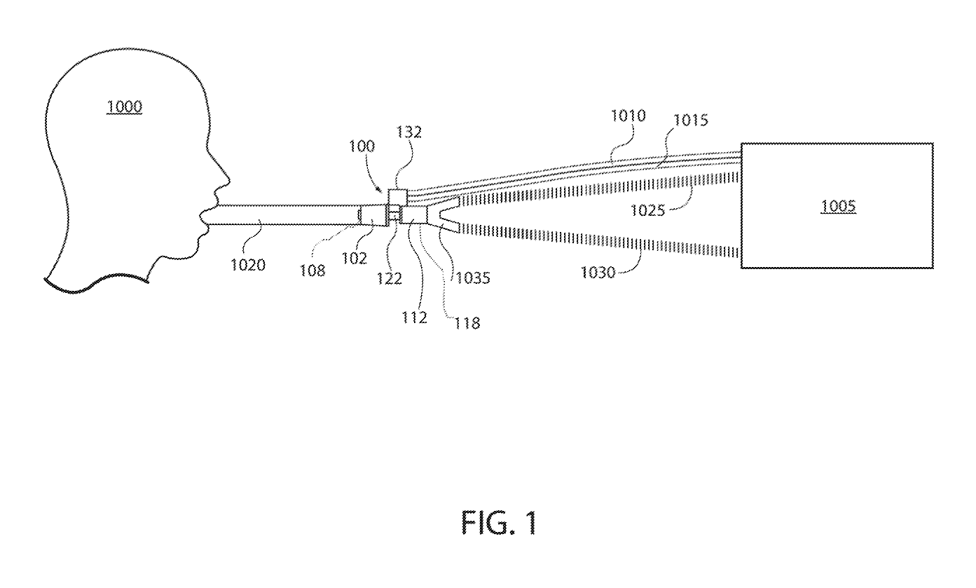

FIG. 1 is a schematic view of a respiratory system using a neonatal flow sensor. While examples described herein may be referred to as neonatal flow sensors, it is to be understood that the flow sensors may find use with other patients, including adult patients, in other examples. FIG. 1 shows a patient 1000; ventilator 1005; a patient respiration connector 1020; a flow sensor 100 with a patient end 108, a first tubular body 102, a ventilator end 118, a second tubular body 112, a third tubular body 122 and a connector 132; a first flow measurement tube 1010; a second flow measurement tube 1015; a expiratory limb 1025; an inspiratory limb 1030; and a Y-connector 1035. In an example, the flow sensor 100 is placed in line with the ventilator 1005 and the patient respiration connector 1020 to fluidly connect the ventilator 1005 and the patient respiration connector 1020. In an example, the patient respiration connector may be a mask connected to the patient's mouth, nose, both the mouth and nose, an endotracheal tube, or the like.

A first flow measurement tube 1010 and a second flow measurement tube 1015 may extend from the flow sensor 100, the first flow measurement tube 1010 aligned with and fluidly connected to the connector 132, and the second flow measurement tube 1015 aligned with and fluidly connected to the connector 132. In an example, the flow measurement tubes 1010, 1015 are flexible hoses.

The second tubular body 112 with a ventilator end 118 may be connected to a Y-connector 1035, which may be fluidly connected to the expiratory limb 1025 and the inspiratory limb 1030. Both the expiratory limb 1025 and the inspiratory limb 1030 are fluidly connected to the ventilator 1005.

The first flow measurement tube 1010 and the second flow measurement tube 1015 may be fluidly connected with a system electrically connected and/or fluidly connected with the ventilator 1005 or an intermediate system to measure a differential pressure or velocity of a gaseous fluid flowing through the flow sensor 100. In an example, the connection of the tubes 1010, 1015 to the flow sensor 100 shifts the center of mass of the flow sensor 100 and the tubes 1010, 1015 towards the ventilator 1005, moving the weight away from the patient 1000 and towards the ventilator 1005 which may increase patient comfort.

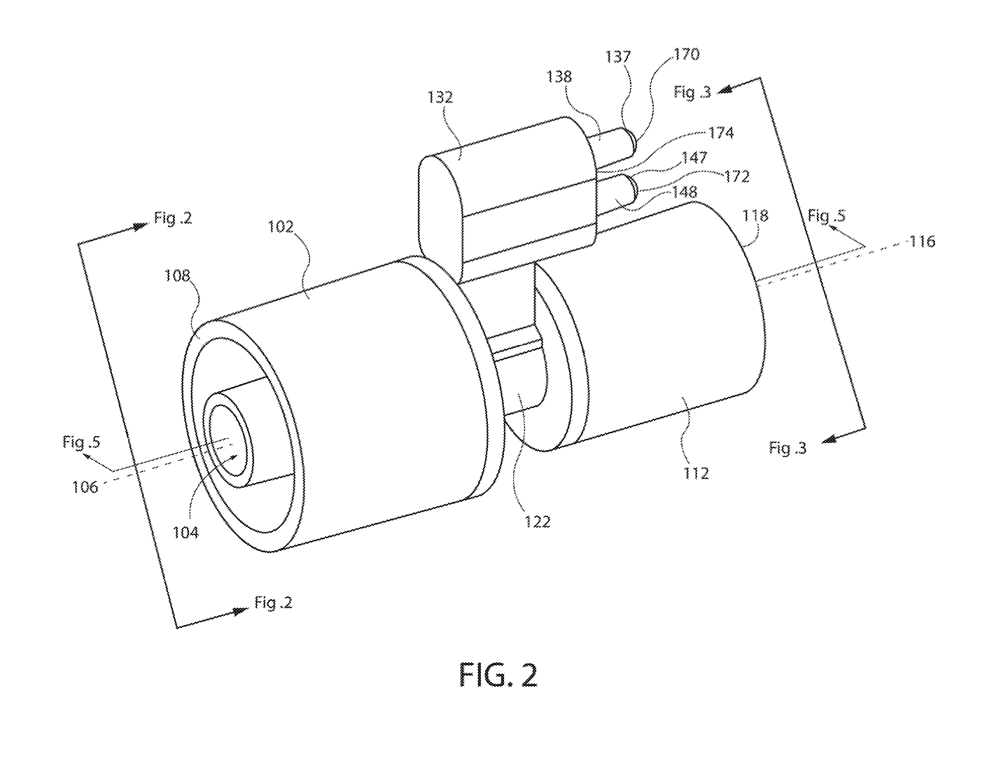

FIG. 2 is a perspective view of an example neonatal flow sensor. The flow sensor 100 may have a first tubular body 102 having a first bore 104, a central axis 106, and a patient end 108; a second tubular body 112 with a central axis 116 and a ventilator end 118; a third tubular body 122; a connector 132 with a first lumen 138 having a second end 137 and a nipple 170; a second lumen 148 having a second end 147 and a nipple 172; and a ventilator proximate surface 174.

In an example, the flow sensor 100 of FIG. 2 may facilitate neonatal respiration measurement. The flow sensor 100 may be made up of at least one tabular body. In an example, the flow sensor 100 may have a first tubular body 102 configured to fluidly connect to the patient respiration connector 1020 of FIG. 1, for example through an endotracheal tube or a mask, at a patient end 108; a second tubular body 112 configured to connect to a ventilation system at a ventilator end 118, and a third tubular body 122 that connects the first tabular body 102 and the second tubular body 112.

The flow sensor 100 may have a connector 132 configured to connect to tubing or flexible hoses that may be fluidly connected to a measurement system that, calculates the amount of air flow through the flow sensor 100. In an example, the connector 132 is located adjacent a third tubular body 122 and between the first, and second tubular bodies 102, 112. The connector 132 may have nipples 170, 172 configured to connect with tubing or flexible hoses, for example the first flow measurement tube 1010 and the second flow measurement tube 1015 shown in FIG. 1. The nipples 170, 172 may extend from a ventilator proximate surface 174 of the connector 132. The nipples 170, 172 may be vertically aligned with each other such that they sit above a portion of the second tubular body 112. In an example, the nipples 170, 172 are parallel, with the nipple 170 located vertically above nipple 172.

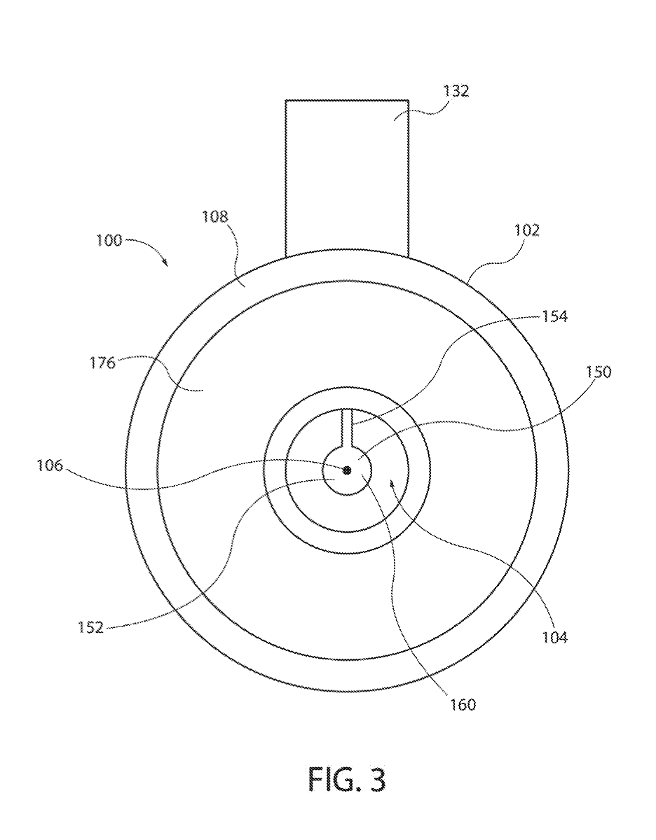

FIG. 3 is a front elevation view of the How sensor of FIG. 2, FIG. 3 shows a flow sensor 100 with a first tubular body 102 having a first bore 104, a patient end 108, a central axis 106, and an annular groove 176; and a restrictor 150 with a post 152 having a ventilator end 160, and a shaft 154.

The patient end 108 of the flow sensor 100 is shown in FIG. 3. In an example, the patient end 108 of the first tubular body 102 may be configured with an annular groove 176 configured to enable the flow sensor 100 to connect to the patient respiration connector 1020, shown in FIG. 1. The annular groove 176 may surround the bore 104, and the bore 104 may have a central axis 106.

In an example, the restrictor 150 may have a post 152 and a shaft 154. A patient end 160 of the post 152 may be seen through the first bore 104. In an example, the post 152 may be cylindrically shaped, with the shaft 154 vertically extending from the post 152. In an example, the shaft 154 may be rectangular shaped. The central axis 106 of the first tubular body may coincide with or be parallel to the post 152. In an example, the post 152 may be shaped as a cylinder, rectangle, oblong, air foil, or other geometries. In an example, the shaft 154 may be shaped as a cylinder, rectangle, oblong, air foil, or other geometries.

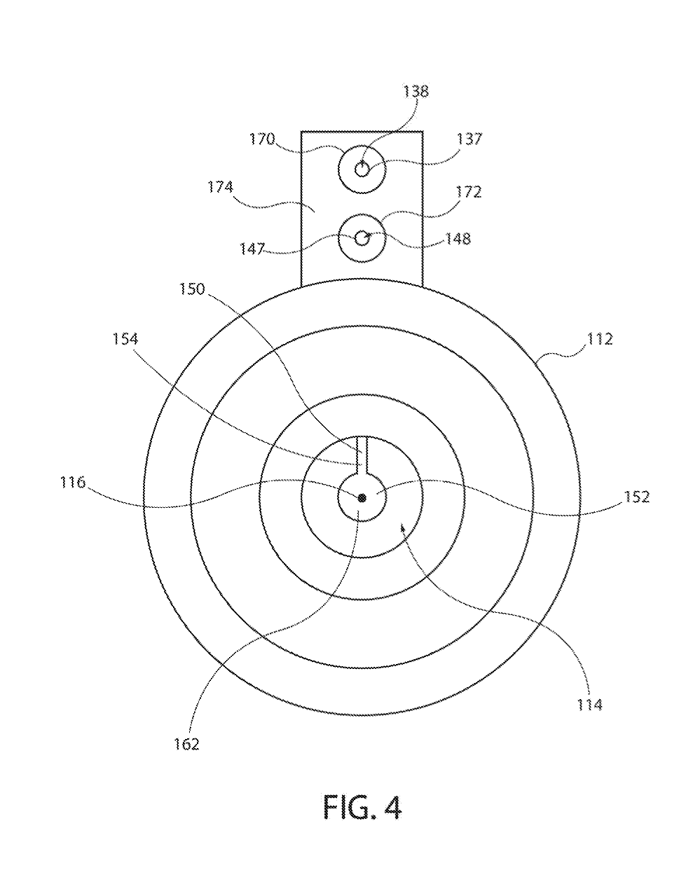

FIG. 4 is a rear elevation view of the flow sensor of FIG. 2. FIG. 4 shows a flow sensor 100 with a second tubular body 112 having a second bore 114, a ventilator end 118, and a central axis 116; a restrictor 150 having a post 152 with a ventilator end 162 and a shaft 154; a connector 132 with a ventilator proximate surface 174, a first lumen 138 with a second end 137 and a nipple 170; and a second lumen 148 with a second end 147 and a second nipple 172.

The ventilator end 118 of the flow sensor 100 is shown in FIG. 4. In an example, the restrictor 150 may have a post 152 with a ventilator end 162 and a shaft 154 visible through the bore 114. In an example, the shape of the post 152 and shaft 154 are similar to the restrictor 150 shown in FIG. 3, The ventilator end 118 may connect to the ventilator 1005 shown in FIG. 1, such as a mechanical ventilation system configured to assist in inhalation and expiration of a patient's breathing. In an example, the ventilator end 118 may be shaped with various annular grooves or features to correspond with common adaptors or neonatal mechanical ventilation systems.

The connector 132 may have a ventilator proximate surface 174 from which a first lumen 138 and a second lumen 148 extend. In FIG. 4, the second end 137 of the first lumen 138 and the second end 147 of the second end lumen 148 are shown. In an example, each second ends 137, 147 of the lumens 138, 148 may be shaped as nipples 170, 172 configured to connect to tubing or flexible hoses. In an example, the second end 137 of the first lumen 138 is vertically adjacent the second end 147 of the second lumen 148.

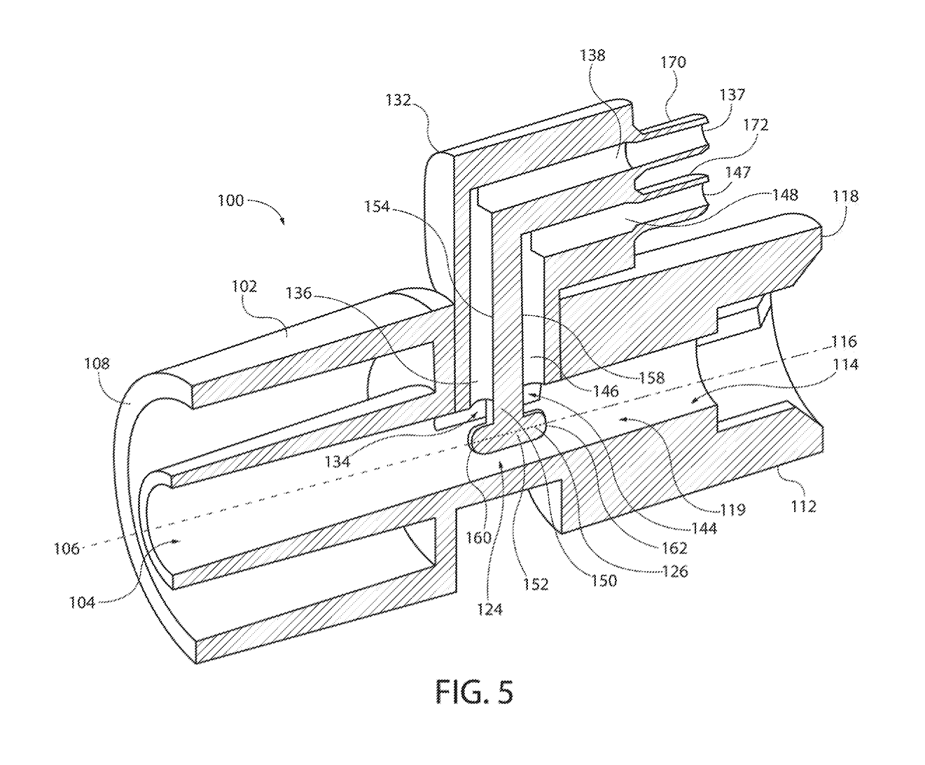

FIG. 5 is a cross-sectional isometric view of the neonatal flow sensor of FIG. 2 along line 5-5. FIG. 5 shows flow sensor 100 with a connector 132 having a first pressure port 134, first lumen 138 having a first end 136 and a second end 137; a second pressure port 144, a second, lumen 148 having a first end 146 and a second end 147; a shaft 154 having a first surface 156 and a second surface 158; a post having a patient end 160 and a ventilator end 162; and a continuous central bore 119.

In an example, the connector 132 of the flow sensor 100 has a first lumen 138 and a second lumen 148. Each lumen has a first end and a second end. In an example, the first lumen 138 has a first end 136 and a second end 137, and the second lumen 148 has a first end 146 and a second end 147. In an example, the first end 136 of the first lumen 138 is generally perpendicular to the second end 137 of the first lumen 138, and the first end 146 of the second lumen 148 is generally perpendicular to a second end 147 of the second lumen 148. In an example, both lumens 138, 148 may have generally constant widths and the first lumen 138 may be generally parallel with the second lumen 148 such that the first end 136 may be generally parallel with the first end 146, and the second end 137 may be generally parallel with the second end 147.

The shaft 154 is located between the parallel portions of the first end 136 of first lumen 138 and the first end 146 of second lumen 148. The shaft 154 extends between the first lumen 138 and the second lumen 148 until the lumens 138, 148 generally perpendicularly turn into the direction of the ventilator proximate surface 174 (shown in FIG. 4). In an example, the shaft 154 has a width equal to the distance between the first and the second pressure ports 134, 144. In an example, a distance between the second ends 137, 147 of the first and second lumens 138, 148 is greater than the width of the shaft 154.

The continuous central bore 119 may fluidly connect with the first lumen 138 at a first pressure port 134 and fluidly connect with the second lumen 148 at the second pressure port 144. In an example, the first surface 156 of the shaft 154 forms a portion of the first pressure port 134, and the second surface 158 forms a portion of the second pressure port 144.

In an example, the post 152 may be aligned with the continuous central bore 119 and extends the distance between the patient end 160 and the ventilator end 162. The length of the post 152 may be equal to a combination of a diameter of the first pressure port 134, a diameter of the second pressure port 144, and the width of the shaft 154. In an example, the patient end 160 of the post 152 is aligned with the first pressure port 134, and the ventilator end 162 of the post 152 is aligned with the second pressure port 144. In an example, the post 152 may he shaped as a cylinder, rectangle, oblong, air foil, or other geometries.

In an example, the flow sensor 100 has a first tubular body 102 having a first bore 104, a first, central axis 106, and a patient end 108 configured to be in fluid communication with a patient 1000; a second tubular body 112 having a second bore 114, a second central axis 116, and a ventilator end 118 configured, to be in fluid communication with a ventilator 1005; a third tubular body 122 having a third bore 124 and a third central axis 126, wherein the third bore 124 is adjacent both the first bore 104 and the second bore 114 to form a continuous central bore 119 extending from the patient end 108 to the ventilator end 118; and a connector 132 including a first pressure port 134 adjacent the first tubular body 102 and in fluid communication with a first end 136 of a first lumen 138; a second pressure port 144 adjacent the second tubular body 112 and in fluid communication with a first end 146 of a second lumen 148; and a restrictor 150 having a post 152 and a shaft 154, the shaft 154 having a width equal to a spacing between the first and the second pressure ports 134, 144 and the post 152 aligned with the continuous central bore 119; wherein the first end 136 of the first lumen 138 is generally perpendicular to a second end 137 of the first lumen 138, and the first end 146 of the second lumen 148 is generally perpendicular to a second end 147 of the second lumen 148.

In an example, the second end 137 of the first lumen 138 is vertically adjacent the second end 147 of the second lumen 148. In an example, the first end 136 of the first lumen 138 is adjacent the first tubular body 102, and the first end 146 of the second lumen 148 is adjacent the second tubular body 112, wherein the first ends 136, 146 of the first and second tubular bodies 102, 112 are aligned with the restrictor shaft 154. In an example, a first surface 156 of the shaft 154 forms a portion of the first pressure port 134 and a second surface 158 of the shaft 154 forms a portion of the second, pressure port 144. In an example, the shaft 154 may be shaped as a cylinder, rectangle, oblong, air foil, or other geometries.

In some examples, the post 152 has a patient end 160 located beneath the first pressure port 134, and a ventilator end 162 located beneath the second pressure port 144, and the post 152 length is a distance between the patient end 160 and the ventilator end 162; and the post 152 length is equal to a combination of a diameter of the first pressure port 134, a diameter of the second pressure port 144 and the width of the shaft 154. In an example, the continuous central bore 119 is sloped to promote drainage ventilator end 118.

In some examples, a flow sensor 100 may have a tubular body 102 or 112 with a bore 104 or 114, a ventilator end 118, a patient end 108, and a central region/tubular body 122; a restrictor 150 located adjacent the central region/tubular body 122 and fluidly connected to the bore 104 or 114, the restrictor 150 having a first pressure port 134 having a first diameter, proximate the patient end, and fluidly connected to a second portion 137 of a first lumen 138, a second pressure port 144 having a second diameter, proximate the ventilator end 118, and fluidly connected to a second portion 147 of a second lumen 148; a restrictor post 152 connected to a shaft 154, the restrictor post 152 having a length that is generally equal to a sum of the first diameter, the second diameter, and a width of the shaft 154; wherein a first portion for example end 136, of the first lumen 138 is generally perpendicular to a second portion, for example end 137, of the first lumen 138, and a first portion, for example end 146, of the second lumen 148 is generally perpendicular to a second portion, for example end 147, of the second lumen 148.

In some examples, the first portion, for example end 136, of the first lumen 138 is adjacent the first pressure port 134 and the second portion, for example end 137, of the first lumen 138 is proximate to the ventilator end 118; and the first portion, for example end 146, of the second lumen 148 is adjacent the second pressure port 144 and a second portion, for example end 147, of the second lumen 148 is proximate to the ventilator end 118. In some examples, the second end 137 of the first lumen 138 is vertically adjacent the second end 147 of the second lumen 148. In some examples, a distance between the second ends 137, 147 of the first and second lumens 138, 148 is greater than the width of the shaft 154. In an example, the second ends 137, 147 of the first and second lumens 138, 148 are shaped as nipples 170, 172.

In some examples, the ventilator end 118 of the flow sensor 100 is configured to connect to the ventilator 1005. In some examples, the patient end 108 of the flow sensor 100 is configured to connect to the patient respiration connector 1020. In some examples, the restrictor post 152 is cylindrically shaped. In some examples, the continuous central bore 119 of the tubular body is sloped to promote drainage.

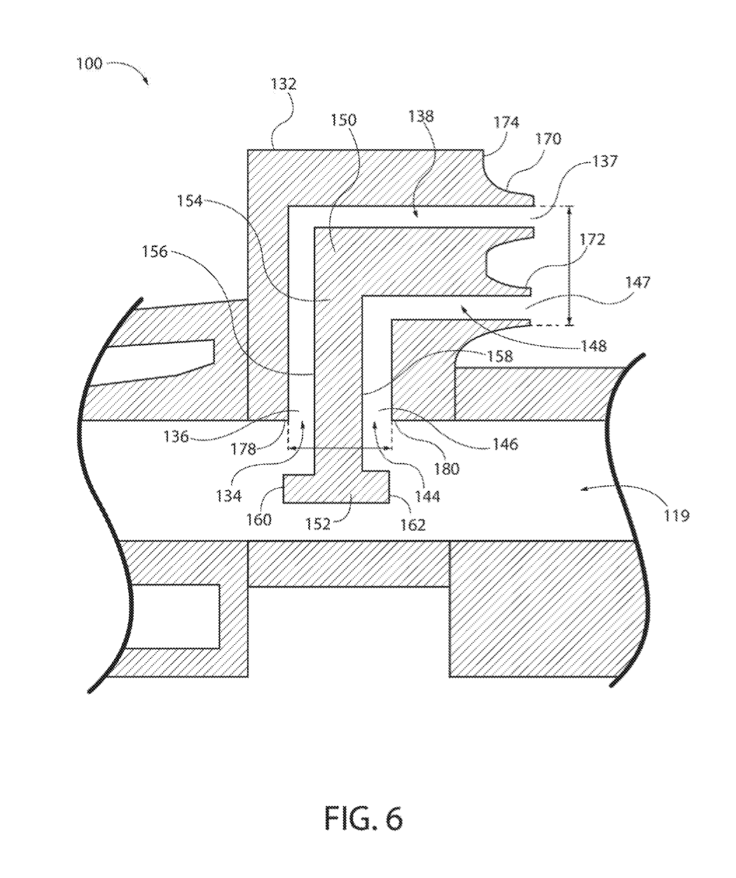

FIG. 6 is an enlarged cross sectional right side view of the flow sensor of FIG. 5. FIG. 6 shows an enlarged sectional view of a flow sensor 100 having a connector 132; a first lumen 138 with a first end 136 and a second end 137; a second lumen 148 with a first end 146 and a second end 147; a first pressure port 134 with a patient edge 178; a second pressure port 144 with a ventilator edge 180; a restrictor 150 with a post 152 having a patient end 160 and a ventilator end 162, a shaft 154 having a first surface 156 and a second surface 158; a first nipple 170; a second nipple 172; a ventilator proximate surface 174; and a continuous central bore 119.

Similar to FIG. 5, FIG. 6 shows a cross-sectional view of a flow sensor 100 where the post 152 has a patient end 160 aligned with the first pressure port 134 and a ventilator end 162 aligned with the second pressure port 144. In an example, a patient edge 178 of the first pressure port 134 is aligned in an approximate same vertical plant as the patient end 160 of the post 152. In an example, a ventilator edge 180 of the second pressure port 144 is aligned in an approximate same vertical plane as the ventilator end 162 of the post 152.

In general, the connector 132 connects a portion of the first tubular body 102 and a portion of the second tubular body 112. In an example, the connector 132 may have a center of mass that is biased towards to the second tubular body 112. The post 152 may be generally concentrically aligned with the central bore 119, and the second, ends 137, 147 of the lumens 138, 148 may be generally parallel with the continuous central bore 119 and/or the post 152.

In an example, the patient end 160 of the post 152 may be generally flat with a face or edges generally parallel to the shaft 154. In other examples, the patient end 160 may be curved and/or feature rounded and/or smooth edges. In an example, the ventilator end 162 of the post 152 may be generally flat with a face or edges parallel to the shaft 154. In other examples, the ventilator end 162 may he curved and/or feature rounded and/or smooth edges.

In some examples, a neonate flow sensor 100 may have a connector 132 having a first lumen 138 with a first end 136 fluidly connected to a first pressure port 134 and a second end 137 generally perpendicular to the first lumen's first end 136; a second lumen 148 with a first end 146 fluidly connected to a second pressure port 144 and a second end 147 generally perpendicular to the second lumen's first end 146; a restrictor 150 having a shaft 154 connected to a post 152, the shaft 154 located between the first lumen's first end 136 and the second lumen's first end 146; and a tubular body with a patient end 108, a ventilator end 118, and a continuous central bore 119, all fluidly connected to the first and second lumens 168,148, and the second end 137 of the first lumen 138 and the second end 147 of the second lumen 148 are proximate the ventilator end 118.

In some examples, a neonate flow sensor 100 may have a continuous central bore 119 sloped to promote drainage. In some examples, a neonate flow sensor 100 may have a diameter of the first pressure port 134 that is generally equal a diameter of the first lumen 138, and a diameter of the second pressure port 144 that is generally equal to a diameter of the second lumen 148. In some examples, a neonate flow sensor 100 may have a restrictor 150 with a post 152 with a length equal to a combined length of the diameter of the first lumen 138, the diameter of the second lumen 148, and a width of the shaft 154. In some examples, the post 152 may have length that is longer than the combined length of the diameter of the first lumen 138, the diameter of the second lumen 148, and a width of the shaft 154. In some examples, the post 152 may have a length that is shorter than the combined length of the diameter of the first lumen 138, the diameter of the second lumen 148, and a width of the shaft 154. In some examples, a neonate flow sensor 100 may have the second end 137 of the first lumen 138 and the second end 147 of the second lumen 148 configured to attach tubing for measuring a neonate patient's respiration.

The flow sensor 100 device disclosed in FIGS. 1-6 may have a unique shape based upon the position of the first lumen 138 and second lumen 148 within the connector 132. The first ends 136, 146 may be generally perpendicular to the second ends 137, 147 and allow the center of the mass of the restrictor 150, and therefore the flow sensor 100, to be biased towards the ventilator end 118 of the flow sensor 100, as opposed to be centered above the post 152. The shift of the center of mass may increase patient comfort, help with alignment of the flow sensor 100 with reference to the ventilator 1005 and patient, respiration system 1020, and allow the accuracy of the flow sensor 100 to Increase when measuring gaseous flow through the flow sensor 100 so patient fluids will drain away from the post 152.

The compact design of the flow sensor 100 may be partially derived from the alignment of the first lumen 138 and the first pressure port 134 as well as the second lumen 148 and the second pressure port 144 with respect to the post 152 and the shaft 154 of the restrictor 150. In addition, the compact design may also allow the flow sensor 100 to be placed in a closer proximity to the patient 1000, which may increase the accuracy of the flow sensor 100 since it may more quickly measure the inhalations and exhalations of the pattern 1000 and decrease the additional circuit volume dead space between the patient 1000 and the Y-connector 1035 through the addition of the flow sensor 100. This may be desirable as neonate patients have very small lung capacities, and there is a volume of air that may never clear the area that exists between the patient 1000 and the Y-connector 1035. This volume of air is being constantly inhaled and exhaled by the patient 1000, each time being further depleted of oxygen. The minimization of the volume of depleted air may benefit the patient 1000 so that the patient 1000 breathes a larger or the largest quantity of fresh, oxygen rich air available. In an example, the volume of depleted air inhaled by the patient 1000 may be minimized by using a flow sensor 100 with a small footprint. In an example, the footprint of the flow sensor 100 may be decreased by designing the layout of the flow sensor 100 with a minimized distance between the pressure ports 134, 144. In an example, the close proximity of the pressure ports 134, 144 to each other enables the overall footprint of the flow sensor 100 to be minimized.

The flow sensor 100 may also increase the accuracy of flow measurement because of the layout of the pressure ports 134, 144. In an example, the pressure ports 134, 144 are located directly above the post 152, which may allow for a more accurate collection of the gaseous fluid within the flow sensor 100. In addition, the location of the pressure ports 134, 144 in relation to the post 152 may help more accurately determine the disruption of the flow caused by the flow being forced around the restrictor post 152. This disruption is used to calculate the overall volume of flow within the flow sensor 100. Neonate patients may have very shallow, low flow breathing, so accurate flow measurement is very important.

In an example, the flow sensor 100 may also increase the accuracy in some examples of flow measurement by draining moist excretions that may coat the inside of the flow sensor 100 and undesirably disrupt the flow measurement. The continuous central bore 119 may be sloped to help with drainage of liquids within the flow sensor 100. In an example, the bore is sloped towards the ventilator end 118 of the flow sensor 100 to allow patient mucus to drain away from the patient 1000 and be collected past the Y-connector 1035. A sloped continuous bore may help to ensure that liquid, such as mucus or condensation, is not surrounding or coating the post 152 such that a flow measurement of gaseous flow around the post 152 would be negatively affected, and it may also increase patient comfort, so that excess mucus isn't draining back towards the patient.

All relative and directional references (including: upper, lower, upward, downward, left, right, leftward, rightward, top, bottom, side, above, below, front, middle, back, vertical, horizontal, and so forth) are given by way of example to aid the reader's understanding of the particular embodiments described herein. They should not be read to be requirements or limitations, particularly as to the position, orientation, or use unless specifically set forth in the claims. Connection references (e.g., attached, coupled, connected, joined, and the like) are to he construed broadly and may include intermediate members between a connection of elements and relative movement between elements. As such, connection references do not necessarily infer that two elements are directly connected and in fixed relation to each other unless specifically set forth in the claims.

Those skilled in the art will, appreciate that the presently disclosed embodiments teach by way of example and not by limitation. Therefore, the matter contained in the above description or shown in the accompanying drawings should be interpreted as illustrative and not in a limiting sense. Thus, it is intended that the scope of the present disclosure should not be limited by the particular embodiments described above. Changes in detail or structure may be made without departing from the spirit of the invention as defined in the appended claims. In methodologies directly or indirectly set forth herein, various steps and operations are described in one possible order of operation, but those skilled in the art will recognize that steps and operations may be rearranged, replaced, or eliminated without necessarily departing from the spirit and scope of the present disclosure as claimed below.

What is claimed is:

1. A flow sensor comprising:
a first tubular body having a first bore, a first central axis, and a patient end configured to be in fluid communication with a ventilator; a second tubular body having a second bore, a second central axis, and a ventilator end configured to be in fluid communication with the ventilator;
a third tubular body having a third bore and a third central axis, wherein the third bore is adjacent both the first bore and the second bore to form a continuous central bore extending from the patient end to the ventilator end;
a connector comprising:
a first, pressure port adjacent the first tubular body and in fluid communication with a first end of a first lumen;
a second pressure port adjacent the second tubular body and in fluid communication with a first end of a second lumen; and
a restrictor having a post and a shaft, the shaft having a width equal to a spacing between the first and the second pressure ports and the post having a length that, is aligned with the continuous central bore;
wherein the first end of the first lumen is perpendicular to a second end of the first lumen, and the first end of the second lumen is perpendicular to a second end of the second lumen.

2. The flow sensor of claim 1, wherein the second end of the first lumen is vertically adjacent the second end of the second lumen.

3. The flow sensor of claim 1, wherein the first end of the first lumen is adjacent the first tubular body, and the first end of the second lumen is adjacent the second tubular body, wherein the first ends of the first and second tubular bodies are aligned with the shaft of the restrictor.

4. The flow sensor of claim 1, wherein a first surface of the shaft forms a portion of the first pressure port and a second surface of the shaft forms a portion of the second pressure port.

5. The flow sensor of claim 1, wherein the post comprises a patient end located beneath the first pressure port, and a ventilator end located beneath the second pressure port, and the post length is a distance between the patient end and the ventilator end; the post length is equal to a combination of a diameter of the first pressure port, a diameter of the second pressure port and the width of the shaft.

6. The flow sensor of claim 5, wherein the continuous central bore is sloped to promote drainage.

7. A flow sensor comprising:
a tubular body having a bore, a ventilator end, a patient end, and a central region;
a restrictor assembly located adjacent the central region and fluidly connected to the bore the restrictor assembly having
a first pressure port having a first diameter, proximate the patient end, and fluidly connected to a second portion of a first lumen;
a second pressure port having a second diameter, proximate the ventilator end, and fluidly connected to a second portion of a second lumen; and
a restrictor post having a length connected to a shaft having a width;
wherein a first portion of the first lumen is perpendicular to the second portion of the first lumen, and a first portion of the second lumen is perpendicular to the second portion of the second lumen.

8. The flow sensor of claim 7, wherein:
the first portion of the first lumen is adjacent the first pressure port and the second portion of the first lumen is proximate to the ventilator end; and
the first portion of the second lumen is adjacent the second pressure port and the second portion of the second lumen is proximate to the ventilator end.

9. The flow sensor of claim 8, wherein the second portion of the first lumen is vertically adjacent the second portion of the second lumen.

10. The flow sensor of claim 9, wherein a distance between the second portions of the first and second lumens is greater than the width of the shaft.

11. The flow sensor of claim 9, wherein the second portions of the first and second lumens are shaped as nipples.

12. The flow sensor of claim 7, wherein the ventilator end is configured to connect to a ventilator.

13. The flow sensor of claim 7, wherein the patient end is configured to connect to a patient respiration connector.

14. The flow sensor of claim 7, wherein the restrictor post is cylindrically shaped.

15. The flow sensor of claim 7, wherein the bore is sloped to promote drainage.

16. A neonate flow sensor comprising:
a connector having
a first lumen with a first end fluidly connected to a first pressure port and a second end perpendicular to the first lumen's first end;
a second lumen with a first end fluidly connected to a second pressure port and a second end perpendicular to the second lumen's first end; and
a restrictor having a shaft connected to a post, the shaft located between the first lumen's first end and the second lumen's first end; and
a tubular body with a patient end, a ventilator end, and a bore, all fluidly connected to the first and second lumens, and the second end of the first lumen and the second end of the second lumen are proximate the ventilator end.

17. The neonate flow sensor of claim 16, wherein the bore is sloped to promote drainage toward the ventilator end.

18. The neonate flow sensor of claim 16, wherein a diameter of the first pressure port is equal a diameter of the first lumen, and a diameter of the second pressure port is equal to a diameter of the second lumen.

19. The neonate flow sensor of claim 16, wherein the post of the restrictor has a length equal to a combined length of the diameter of the first lumen, the diameter of the second lumen, and a width of the shaft.

20. The neonate flow sensor of claim 16, wherein the second end of the first lumen and the second end of the second lumen are configured to attach tubing for measuring a neonate patient's respiration.
